(19) United States
(12) Patent Application Publication
(10) Pub. No.: US 2025/0361484 A1
(43) Pub. Date: Nov. 27, 2025

(54) USE OF MEMORY LYMPHOCYTE POPULATION IN LIVER CANCER TREATMENT

(71) Applicant: CYTOCRAFT BIOPHARMACEUTICAL CO., LTD., Chengdu (CN)

(72) Inventors: Ligong CHEN, Beijing (CN); Zhongjie SUN, Beijing (CN); Hong ZHAO, Beijing (CN); Jianqiang CAI, Beijing (CN); Junfan MA, Beijing (CN); Xiao GUO, Beijing (CN); Hailong QI, Beijing (CN); Qinglei XUE, Beijing (CN); Shuren ZHANG, Beijing (CN)

(21) Appl. No.: 19/292,957

(22) Filed: Aug. 7, 2025

Related U.S. Application Data
(63) Continuation of application No. 17/209,268, filed on Mar. 23, 2021.

(51) Int. Cl. C12N 5/0783; A61K 40/11; A61K 40/42; A61P 35/00

(52) U.S. Cl. CPC C12N 5/0636; A61K 40/11; A61K 40/42; A61P 35/00; A61K 2239/38; A61K 2239/53; C12N 2501/2302; C12N 2501/2307; C12N 2501/2315; C12N 2501/515; C12N 2506/11

(57) ABSTRACT

Provided is a method for treating liver cancer. The method includes administrating a therapeutic effective amount of a memory lymphocyte population to a subject in need thereof. The memory lymphocyte population contains at least one of the following marker molecules: leukocyte differentiation antigens CD3, CD4, CD8, CD16, CD56, CD62L and CD45RO.

### CROSS-REFERENCE TO RELATED APPLICATIONS

The present application is a continuation of U.S. application Ser. No. 17/209,268, filed on Mar. 23, 2021, which is a continuation of International Application No. PCT/CN2019/074912, filed on Feb. 13, 2019, and which claims a priority to Chinese Patent Application No. 201910068937.5, filed on Jan. 24, 2019. The disclosures of the aforementioned applications are hereby incorporated by reference in their entireties.

### FIELD

The present disclosure relates to the field of biology. Specifically, the present disclosure relates to a method for treating liver cancer.

### BACKGROUND

Cancer is a category of diseases in which cells proliferate malignantly and invade normal tissues of a body. It has a high incidence, and more than 14.6% of deaths are caused by cancer each year. The battle to conquer cancer has never stopped, and there are continuous new developments. However, there is still no perfect cure for cancer.

The most common method of cancer treatment is surgical resection, followed by radiotherapy and chemotherapy. But all three treatments have certain defects, such as the inability to remove all cancer cells, causing residual cancer cells to invade adjacent tissues or metastasize remotely and failure, or damage of normal tissues while killing cancer cells. Since the 1990s, people have developed targeted therapies with cell-specific recognition capabilities, that is, therapies using monoclonal antibody drugs that specifically recognize cancer cell antigens or tyrosine phosphokinase inhibitors that block intracellular signal transmission in cancer cells to accurately kill cancer cells. The targeted therapy, because of its cancer cell specificity, avoids side effects caused by the complete destruction of traditional chemotherapy. Generally, in an early stage of cancer, surgery or radiation therapy can be used to reduce the number of cancer cells, and then, targeted therapy, drug chemotherapy, or a combined treatment thereof at different stages can be adopted. Although the combined treatment has good curative effects, it still cannot completely cure all cancers, and patients often have cancer recurrence. Therefore, new treatment methods need to be explored. The invention of adoptive immune cell therapy provides people with new ideas.

The adoptive immune cell therapy is a treatment that transfers donor's lymphocytes to recipients to enhance their cellular immune function, and it has a wide range of applications and prospects in anti-cancer, hematopoietic stem cell transplantation, anti-viral infection and autoimmune therapy.

Cell products used in T-lymphocyte-based adoptive immune cell therapy mainly include TIL, TCR-T and CAR-T. Although TIL, TCR-T and CAR-T have certain effects in tumor treatment, their survival rates are still not high, and thus, they are not universal. The bottleneck of the development of TIL, TCR-T and CAR-T mainly lies in the in vivo cytokine storm triggered by them and the transient survival of effector T cells in vivo. Generally, highly differentiated effector T cells, such as cytotoxic T cells, have a half-life period of only about 15 days in vivo. The survival time of memory T cells, including effector memory T cells (TEM) and central memory T cells (TCM), with strong stemness can be prolonged to one month or more, and the effect of killing tumors is better.

### SUMMARY

According to an aspect, the present disclosure provides a method for treating liver cancer. The method includes administrating a therapeutic effective amount of a memory lymphocyte population to a subject in need thereof. The memory lymphocyte population contains at least one of the following marker molecules: leukocyte differentiation antigens CD3,CD4, CD8, CD16, CD56, CD62L and CD45RO.

According to another aspect, the present disclosure provides a method for treating liver cancer in assistance with tumor resection operation. The method includes administrating a therapeutic effective amount of a memory lymphocyte population to a who has undergone radical hepatectomy; and subjecting the subject to TACE therapy.

### BRIEF DESCRIPTION OF DRAWINGS

These and other aspects and advantages of embodiments of the present disclosure will become apparent and more readily appreciated from the following descriptions with reference to the accompanying drawings, in which:

FIG. 1 is a schematic diagram of an analysis of molecular identification on a surface of memory lymphocytes according to an embodiment of the present disclosure;

FIG. 2 is a schematic diagram of an analysis of a proportion of CD39⁺ tumor-specific T cells and a proportion of PD-1⁺ failed T cells in memory lymphocytes according to an embodiment of the present disclosure;

FIG. 3 is a schematic diagram of an analysis of expression changes of CD62L according to an embodiment of the present disclosure, where A is a schematic diagram of an analysis of an expression of CD62L of memory lymphocytes before exposure to an antigen stimulation, and B is a schematic diagram of an analysis of an expression of CD62L of memory lymphocytes after co-culturing with DC cells loaded with liver cancer antigens;

FIG. 4 is a schematic diagram of a cell proliferation analysis of memory lymphocytes stimulated by DC cells loaded with liver cancer tumor antigens according to an embodiment of the present disclosure;

FIG. 5 is a schematic diagram of an analysis of a secretion of an effector cytokine IFN-γ after memory lymphocytes are stimulated by DC cells loaded with liver cancer tumor antigens according to an embodiment of the present disclosure;

FIG. 6 is a tumor image of tumor-bearing nude mice according to an embodiment of the present disclosure, in which, from left to right are representative tumors taken after the mice were sacrificed and dissected at the fifth week;

FIG. 7 is a statistical diagram of a tumor-bearing volume of mice in an experimental group and a control group in the fifth week according to an embodiment of the present disclosure;

FIG. 8 is a schematic diagram of a tumor volume of tumor-bearing nude mice from the first week to the fifth week as a function of time according to an embodiment of the present disclosure;

FIG. 9 is a schematic diagram of an analysis of human CD8⁺ T cell infiltration in tumors borne by mice according to an embodiment of the present disclosure;

FIG. 10A to FIG. 10H illustrate a schematic diagram of an analysis of human TCM retention in peripheral blood of tumor-bearing nude mice according to an embodiment of the present disclosure;

FIG. 11 is a technical roadmap according to an embodiment of the present disclosure;

FIG. 12 is a schematic diagram of a baseline value analysis between a control group and an experimental group in a clinical study related to memory lymphocytes according to an embodiment of the present disclosure, where C represents a percentage of patients corresponding to stage I, II, and III after performing a pathological classification on liver cancer patients according to Edmondson-Steiner grade, and;

FIG. 13 is a disease-free survival curve of interim statistics of a primary efficacy endpoint in a clinical study related to memory lymphocytes according to an embodiment of the present disclosure;

FIG. 14 is an overall survival curve of interim statistics of a secondary efficacy endpoint in a clinical study related to memory lymphocytes and a schematic diagram of an analysis of an expression of AFP according to an embodiment of the present disclosure; and

FIG. 15A and FIG. 15B illustrate a schematic diagram of a safety analysis in a clinical study related to memory lymphocytes according to an embodiment of the present disclosure.

### DESCRIPTION OF EMBODIMENTS

Embodiments of the present disclosure are described in detail below. The embodiments described below are exemplary and only used to explain the present disclosure, and should not be construed as limiting the present disclosure.

In addition, terms such as “first” and “second” are used herein for purposes of description and are not intended to indicate or imply relative importance, or to implicitly show the number of technical features indicated. Thus, the feature defined with “first” and “second” may explicitly or implicitly comprise one or more this feature. Further, in the description of the present disclosure, “a plurality of” means two or more, unless specified otherwise.

The present disclosure provides a memory lymphocyte population, a culture medium, and a method for obtaining the memory lymphocyte population and a use of same, each of which will be described in detail below.

The present disclosure aims to solve at least one of the technical problems in the prior art to a certain extent. To this end, the present disclosure provides a memory lymphocyte population, a culture medium, and a method for obtaining the memory lymphocyte population and a use of same. The memory lymphocyte population according to the present disclosure can be activated, proliferate and differentiate rapidly under specific stimulation of liver cancer antigens, and it can secrete IFN-γ, thereby assisting in strengthening a body immune response.

In addition, since the memory lymphocyte population can be retained in the body, it has a better tumor killing effect. In addition, the memory lymphocyte population can effectively reduce the risk of tumor metastasis and recurrence, and is especially suitable for liver cancer with microvascular invasion.

To this end, according to an aspect of the present disclosure, the present disclosure provides a memory lymphocyte population. According to embodiments of the present disclosure, the memory lymphocyte population contains at least one of the following marker molecules: leukocyte differentiation antigens CD3, CD4, CD8, CD16, CD56, CD62L and CD45RO. Consequently, the memory lymphocyte population according to the embodiments of the present disclosure can be activated, proliferate and differentiate rapidly under specific stimulation of liver cancer antigens, and can secrete IFN-γ, thereby assisting in strengthening the body immune response. In addition, since the memory lymphocyte population can be retained in the body, it has a better tumor killing effect. In addition, the memory lymphocyte population can effectively reduce the risk of tumor metastasis and recurrence, and is especially suitable for liver cancer with microvascular invasion.

According to embodiments of the present disclosure, a main cell population in the memory lymphocyte population is central memory T cells, a content of the main cell population is not less than 70%, and a surface marker molecule of the main cell population is CD3⁺CD45RA⁻CD45RO⁺CD62L⁺.

According to embodiments of the present disclosure, a proportion of CD39⁺PD-1⁻ cell subset in the central memory T cells with a surface marker molecule of CD4⁺ is 5% to 8%.

According to embodiments of the present disclosure, a proportion of CD39⁺PD-1⁻ cell subset in the central memory T cells with a surface marker molecule of CD8⁺ is 35% to 45%.

According to embodiments of the present disclosure, after the memory lymphocyte population is in contact with DC cells loaded with tumor antigens, a proportion of cells with a surface marker molecule of CD62L⁺ in the memory lymphocyte population decreases, a proportion of cells with a surface marker molecule of CD62L⁻ in the memory lymphocyte population increases, and an expression of IFN-γ increases.

According to another aspect of the present disclosure, the present disclosure provides a culture medium. According to embodiments of the present disclosure, the culture medium includes a basal culture medium, interleukin-2, interleukin-7, interleukin-15, an Anti-CD3 antibody, and autologous plasma. The culture medium according to embodiments of the present disclosure can be used to cultivate immune cells, so as to obtain the memory lymphocyte population. The memory lymphocyte population can be activated, proliferate and differentiate rapidly under specific stimulation of liver cancer antigens, and can secrete IFN-γ, thereby assisting in strengthening the body immune response. In addition, since the memory lymphocyte population can be retained in the body, it has a better tumor killing effect. In addition, the memory lymphocyte population can effectively reduce the risk of tumor metastasis and recurrence, and is especially suitable for liver cancer with microvascular invasion.

According to embodiments of the present disclosure, a concentration of the interleukin-2 is 5×10⁴ U/L to 1×10⁶ U/L, and preferably, is 5×10⁵ U/L.

According to embodiments of the present disclosure, a concentration of the interleukin-7 is 1 ng/ml to 60 ng/ml, and preferably 5 ng/mL.

According to embodiments of the present disclosure, a concentration of the interleukin-15 is 1 ng/mL to 60 ng/mL, and preferably 5 ng/mL.

According to embodiments of the present disclosure, a concentration of the Anti-CD3 antibody is 0.5 μg/mL to 10 μg/mL, and preferably 3 μg/mL.

According to embodiments of the present disclosure, a concentration of the autologous plasma is 1 vol % to 10 vol %, and preferably 5 vol %.

According to embodiments of the present disclosure, the basal culture medium is selected from GT-T551 culture medium.

According to embodiments of the present disclosure, the autologous plasma is obtained by centrifuging peripheral blood, collecting supernatant, and inactivating the supernatant at a temperature of 50° C. to 60° C. for 20 to 40 minutes.

According to embodiments of the present disclosure, a pH value of the culture medium is 7.2 to 7.4.

According to yet another aspect of the present disclosure, the present disclosure provides a method for obtaining a memory lymphocyte population. According to embodiments of the present disclosure, the method includes resuspending and culturing naive immune cells in the culture medium as described above, so as to obtain the memory lymphocyte population. The memory lymphocyte population, which is obtained through the method for obtaining the memory lymphocyte population according to embodiments of the present disclosure, can be activated, proliferate and differentiate rapidly under specific stimulation of liver cancer antigens, and can secrete IFN-γ, thereby assisting in strengthening the body immune response. In addition, since the memory lymphocyte population can be retained in the body, it has a better tumor killing effect. In addition, the memory lymphocyte population can effectively reduce the risk of tumor metastasis and recurrence, and is especially suitable for liver cancer with microvascular invasion.

According to embodiments of the present disclosure, the culture medium as described above is supplemented every 2 to 4 days during the culturing, and the culture medium does not contain the autologous plasma in a third supplementation and each of subsequent supplementations.

According to embodiments of the present disclosure, the culture medium is supplemented to a cell density of 5×10⁵ cells/mL to 25×10⁵ cells/mL.

According to embodiments of the present disclosure, the culturing is performed at a temperature of 37° C. and 5 vol % of CO₂ for 10 to 20 days.

According to embodiments of the present disclosure, the naive immune cells are selected from peripheral blood mononuclear cells.

According to embodiments of the present disclosure, the peripheral blood mononuclear cells are resuspended in the culture medium at a density of 5×10⁵ cells/mL to 20×10⁵ cells/mL.

According to embodiments of the present disclosure, before the culturing, a culture container is coated with a coating solution containing the Anti-CD3 antibody at 2° C. to 8° C. for 10 to 16 hours in advance, and a volume of the coating solution is 2 to 8 ml/75 cm² of the culture container.

According to embodiments of the present disclosure, the peripheral blood mononuclear cell is obtained by the following manners: mixing peripheral blood with heparin and centrifuging at 1,200 rpm/min to 2,000 rpm/min for 5 to 10 minutes to obtain an upper layer of autologous plasma and a lower layer of blood cells; and diluting the blood cells with normal saline, loading the diluted blood cells on a surface of a lymphocyte separation solution, centrifuging the lymphocyte separation solution at 1,500 rpm/min to 2,000 rpm/min for 20 to 30 minutes, taking a mononuclear cell layer, mixing the mononuclear cell layer with the normal saline, centrifuging the mononuclear cell layer mixed at 1,500 rpm/min to 2,000 rpm/min for 5 to 10 minutes, and washing the centrifuged mononuclear cell layer for 3 times, so as to obtain the peripheral blood mononuclear cells, in which a volume ratio of the blood cells, the normal saline and lymphocyte separation liquid is (1 to 3):(1 to 3):1.

According to still yet another aspect of the present disclosure, the present disclosure provides a memory lymphocyte population. According to embodiments of the present disclosure, the memory lymphocyte population is obtained by the method for obtaining the memory lymphocyte population as described above. The memory lymphocyte population according to embodiments of the present disclosure can be activated, proliferate and differentiate rapidly under specific stimulation of liver cancer antigens, and can secrete IFN-γ, thereby assisting in strengthening the body immune response. In addition, since the memory lymphocyte population can be retained in the body, it has a better tumor killing effect. In addition, the memory lymphocyte population can effectively reduce the risk of tumor metastasis and recurrence, and is especially suitable for liver cancer with microvascular invasion.

According to still yet another aspect of the present disclosure, the present disclosure provides a use of the memory lymphocyte population as described above in a preparation of a medicine. According to embodiments of the present disclosure, the medicine is used for a treatment of liver cancer. The memory lymphocyte population according to embodiments of the present disclosure can be activated, proliferate and differentiate rapidly under specific stimulation of liver cancer antigens, and can secrete IFN-γ, thereby assisting in strengthening the body immune response. In addition, since the memory lymphocyte population can be retained in the body, it has a better tumor killing effect. In addition, the memory lymphocyte population can effectively reduce the risk of tumor metastasis and recurrence, and is especially suitable for liver cancer with microvascular invasion.

According to embodiments of the present disclosure, the medicine reduces a volume of a tumor tissue.

According to embodiments of the present disclosure, the medicine reduces a content of alpha-fetoprotein in a body administrated with the medicine.

According to embodiments of the present disclosure, the medicine has no hepatic and renal toxicity to a body administrated with the medicine.

Additional aspects and advantages of the present disclosure will be given in the following descriptions, become apparent in part from the following descriptions, or be learned from the practice of embodiments of the present disclosure.

### Memory Lymphocyte Population

In an aspect of the present disclosure, the present disclosure provides a memory lymphocyte population. According to embodiments of the present disclosure, the memory lymphocyte population contains at least one of the following marker molecules: leukocyte differentiation antigens CD3, CD4, CD8, CD16, CD56, CD62L and CD45RO. The memory lymphocyte population according to the present disclosure contains central memory T cells, effect memory T cells, effector T cells, NK cells, and NKT cells, which can be activated, proliferate and differentiate rapidly under specific stimulation of liver cancer antigens, and can secrete IFN-γ, thereby assisting in strengthening a body immune response. In addition, since the memory lymphocyte population can be retained in the body, it has a better tumor killing effect. In addition, the memory lymphocyte population can effectively reduce the risk of tumor metastasis and recurrence, and is especially suitable for liver cancer with microvascular invasion.

According to embodiments of the present disclosure, a main cell population in the memory lymphocyte population is central memory T cells, a content of the main cell population is not less than 70%, and a surface marker molecule of the main cell population is CD3⁺CD45RA⁻CD45RO⁺CD62L⁺. As a result, the memory lymphocyte population can be activated, proliferate and differentiate rapidly under specific stimulation of liver cancer antigens, and can secrete IFN-γ, thereby assisting in strengthening the body immune response. In addition, since the memory lymphocyte population can be retained in the body, it has a better tumor killing effect. In addition, the memory lymphocyte population may effectively reduce the risk of tumor metastasis and recurrence, and is especially suitable for liver cancer with microvascular invasion.

It should be noted that, according to embodiments of the present disclosure, the central memory T cell with the surface marker molecule of CD3⁺CD45RA⁻CD45RO⁺CD62L⁺ further includes two kinds of surface marker molecules: CD4⁺CD3⁺CD45RA⁻CD45RO⁺CD62L⁺ and CD8⁺CD3⁺CD45RA⁻45RO⁺CD62L⁺. A proportion of CD39⁺PD-1⁻ cell subset in the central memory T cells with CD4⁺ is 5 to 8%, and a proportion of CD39⁺PD-1⁻ cell subset in the central memory T cells with CD8⁺ is 35% to 45%. Consequently, the memory lymphocyte population can be activated, proliferate and differentiate rapidly under specific stimulation of liver cancer antigens, and can secrete IFN-γ, thereby assisting in strengthening the body immune response. In addition, since the memory lymphocyte population can be retained in the body, it has a better tumor killing effect. In addition, the memory lymphocyte population can effectively reduce the risk of tumor metastasis and recurrence, and is especially suitable for liver cancer with microvascular invasion.

According to embodiments of the present disclosure, after the memory lymphocyte population is in contact with DC cells loaded with tumor antigens, a proportion of cells with a surface marker molecule of CD62L⁺ in the memory lymphocyte population decreases, a proportion of cells with a surface marker molecule of CD62L⁻ in the memory lymphocyte population increases, and an expression of IFN-γ increases. Consequently, the memory lymphocyte population can be activated, proliferate and differentiate rapidly under specific stimulation of liver cancer antigens, and can secrete IFN-γ, thereby assisting in strengthening the body immune response. In addition, since the memory lymphocyte population can be retained in the body, it has a better tumor killing effect. In addition, the memory lymphocyte population can effectively reduce the risk of tumor metastasis and recurrence, and is especially suitable for liver cancer with microvascular invasion.

### Culture Medium

In an aspect of the present disclosure, the present disclosure provides a culture medium. According to embodiments of the present disclosure, the culture medium includes a basal culture medium, interleukin-2, interleukin-7, interleukin-15, an Anti-CD3 antibody, and autologous plasma.

As a cytokine, interleukin may activate or regulate lymphocytes, and mediate activation and proliferation of the lymphocytes. The Anti-CD3 antibody may specifically recognize CD3 molecules on the surface of T cells, and activate and proliferate the T cells through a combination of TCR-CD3 complexes on the surface of the T cells and MHC-II molecules-antigen peptides on the surface of APCs. The autologous plasma is rich in nutrients and growth factors, which can support cell growth. The inventor compounded the Anti-CD3 antibody, the autologous plasma and various interleukins in the basal culture medium, allowing the memory lymphocyte population in initial immune cells to be activated and proliferated in the system. In addition, a purpose of separation can be achieved, such that the memory lymphocyte population with high purity and good activity can be obtained. In addition, the memory lymphocyte population can be activated, proliferate and differentiate rapidly under specific stimulation of liver cancer antigens, and can secrete IFN-γ, thereby assisting in strengthening the body immune response. In addition, since the memory lymphocyte population can be retained in the body, it has a better tumor killing effect. Also, the memory lymphocyte population can effectively reduce the risk of tumor metastasis and recurrence, and is especially suitable for liver cancer with microvascular invasion.

According to embodiments of the present disclosure, the aforementioned memory lymphocyte population can be obtained by using the culture medium according to embodiments of the present disclosure.

According to embodiments of the present disclosure, a concentration of the interleukin-2 is 5×10⁴ U/L to 1×10⁶ U/L. Therefore, lymphocytes can be effectively activated or regulated, and the activation and proliferation of the lymphocytes can be mediated. The effect is better when the concentration is 5×10⁵ U/L.

According to embodiments of the present disclosure, a concentration of the interleukin-7 is 1 ng/mL to 60 ng/mL. Therefore, the lymphocytes can be effectively activated or regulated, and the activation and proliferation of the lymphocytes can be mediated. The effect is better when the concentration is 5 ng/mL.

According to embodiments of the present disclosure, a concentration of the interleukin-15 is 1-60 ng/ml. Therefore, the lymphocytes can be effectively activated or regulated, and the activation and proliferation of the lymphocytes can be mediated. The effect is better when the concentration is 5 ng/ml.

According to embodiments of the present disclosure, a concentration of the Anti-CD3 antibody is 0.5 μg/mL to 10 μg/mL. Therefore, the lymphocytes can be effectively activated or regulated, and the activation and proliferation of the lymphocytes can be mediated. The effect is better when the concentration is 3 μg/mL.

According to embodiments of the present disclosure, the basal culture medium is selected from GT-T551 culture medium. In this manner, the memory lymphocytes can be better activated and proliferated.

It should be noted that sources of the GT-T551 culture medium, the interleukin-2, the interleukin-7, the interleukin-15 and the Anti-CD3 antibody are not strictly limited in the present disclosure, and can be flexibly selected based on actual conditions. In some embodiments, the GT-T551 culture medium is from Takara Bio Co. Ltd., the interleukin-2 is from Jiangsu Kingsley Pharmaceutical Co. Ltd., the interleukin-7 is from R&D Systems, the interleukin-15 is from R&D Systems, and the Anti-CD3 antibody is from ACROBiosystems. Consequently, the activity and proliferation efficiency of the memory lymphocytes can be further improved.

According to embodiments of the present disclosure, a concentration of the autologous plasma is 1 vol % to 10 vol %. Therefore, the lymphocytes can be effectively activated or regulated, and the activation and proliferation of the lymphocytes can be mediated.

The effect is better when the concentration is 5 vol %.

It should be noted that concentrations of the interleukins, the Anti-CD3 antibody and the autologous plasma in the present disclosure are all limited based on a volume of the basal culture medium.

According to embodiments of the present disclosure, the autologous plasma is obtained by centrifuging peripheral blood, collecting supernatant, and inactivating the supernatant at a temperature of 50° C. to 60° C. for 20 to 40 minutes. After centrifugation, the peripheral blood is divided into an upper layer of plasma and a lower layer of blood cells. The upper layer is inactivated to prepare the culture medium. Mononuclear cells can be separated from the lower layer of blood cells. The mononuclear cells can proliferate in a culture medium containing the autologous plasma. The autologous plasma may provide nutrients for cell proliferation. In addition, due to the same source, an occurrence of rejection is reduced, thereby ensuring a high activity of the memory lymphocyte population obtained. In addition, the inactivated autologous plasma also ensures the safety of the cultured memory lymphocyte population.

According to embodiments of the present disclosure, a pH value of the culture medium is 7.2 to 7.4. The pH value of the culture medium including the basal culture medium, the interleukins, the Anti-CD3 antibody and the autologous plasma is 7.2 to 7.4. Consequently, the memory lymphocytes can be better activated and proliferated.

### Method for Obtaining Memory Lymphocyte Population

In another aspect of the present disclosure, the present disclosure provides a method for obtaining the memory lymphocyte population. According to embodiments of the present disclosure, the method includes, resuspending and culturing naive immune cells in the aforementioned culture medium, so as to obtain the memory lymphocyte population. The memory lymphocyte population obtained by the method for obtaining the memory lymphocyte population according to embodiments of the present disclosure can be activated, proliferate and differentiate rapidly under specific stimulation of liver cancer antigens, and can secrete IFN-γ, thereby assisting in strengthening the body immune response. In addition, since the memory lymphocyte population can be retained in the body, it has a better tumor killing effect. In addition, the memory lymphocyte population can effectively reduce the risk of tumor metastasis and recurrence, and is especially suitable for liver cancer with microvascular invasion. In some embodiments, the method can be used to obtain the memory lymphocyte population as described above.

It should be noted that the term “immune cells” used in the present disclosure refers to cells involved in or related to immune responses, including lymphocytes, dendritic cells, monocytes/macrophages, granulocytes, mast cells, and so on. In some embodiments, the naive immune cells include populations or subpopulations of T cells, NK cells, and/or NKT cells, which can be isolated from human or non-human mammals. Examples of the non-human mammals include, but are not limited to, rabbits, horses, cows, sheep, pigs, dogs, cats, mice, rats, and transgenic species thereof.

The populations or subpopulations of T cells can be obtained or isolated from many sources, including, but not limited to, the peripheral blood, bone marrow, lymph node tissues, umbilical cord blood, thymus tissues, ascites, pleural effusion, spleen tissues, and tumor tissues. The bone marrow can be obtained from the femur, iliac ridge, hip bone, rib, sternum, and other bones.

The populations or subpopulations of NK cells can be obtained or enriched from many sources, including, but not limited to, the peripheral blood, cord blood, and tumors.

Fully mature NKT cells can be obtained or enriched from the peripheral blood. Populations of smaller mature NKT cells can be found in the bone marrow, lymph node tissues, umbilical cord blood, and thymus tissues.

The isolated or enriched populations or subpopulations of T, NK, NKT cells can be obtained from a unit of blood using any number of techniques known to a skilled person (such as Ficoll™ isolation), or obtained through apheresis of T, NK or NKT cells from circulating blood of an individual.

The immune cells may also be tumor infiltrating lymphocytes (TIL) isolated from the tumor tissues.

An initial immune cell population to be cultured may be isolated from a subject or donor, or isolated from or included in the following: the peripheral blood, bone marrow, lymph node tissues, umbilical cord blood, thymus tissues, tissues of an infection site, ascites, pleural effusion, spleen tissues, and tumor separation of the subject/donor. The subject may be healthy, or may suffer from an autoimmune disease, hematopoietic malignancies, a viral infection, or a solid tumor. The subject may be CMV seropositive, or may have been previously administered genetically modified immune cells.

The initial immune cell population to be cultured may be differentiated from stem cells, hematopoietic stem cells or progenitor cells, or from progenitor cells in vitro; or the initial immune cell population to be cultured may be trans-differentiated from non-pluripotent cells of hematopoietic or non-hematopoietic lineage.

According to embodiments of the present disclosure, the aforementioned medium is supplemented every 2 to 4 days during the culturing, in which the culture medium does not contain the autologous plasma in a third supplementation and each of subsequent supplementations. The culture medium is supplemented during the culturing to provide sufficient nutrients for the activation and proliferation of cells. During the first and second supplementations, cells are in an activated state and have a high nutritional requirement, and thus the autologous plasma needs to be supplemented. At this time, the cells are round and bright, and are mainly small cell colonies. In the subsequent supplementations, the cells are in a state of massive proliferation. At this time, the cells are round and bright, and are mainly single cells, and accordingly, the autologous plasma is no longer required to be supplemented.

According to embodiments of the present disclosure, the culture medium is supplemented to a cell density of 5×10⁵ cells/mL to 25×10⁵ cells/mL. Thus, sufficient nutrients and a growth environment can be provided for the memory lymphocytes to promote the activation and proliferation of the memory lymphocytes.

According to embodiments of the present disclosure, the culturing is performed at a temperature of 37° C. and 5 vol % of CO₂ for 10 to 20 days. In this manner, the memory lymphocytes can be better activated and proliferated.

According to embodiments of the present disclosure, the naive immune cells are selected from a peripheral blood mononuclear cell.

According to embodiments of the present disclosure, the peripheral blood mononuclear cell is resuspended in the culture medium at a density of 5×10⁵ cells/mL to 20×10⁵ cells/mL. Consequently, sufficient nutrients and a growth environment can be provided for the memory lymphocytes to promote the activation and proliferation of the memory lymphocytes.

According to embodiments of the present disclosure, before performing the culturing, a culture container is coated with a coating solution containing the Anti-CD3 antibody at 2° C. to 8° C. for 10 to 16 hours in advance, and a volume of the coating solution is 2 to 8 ml/75 cm² of the culture container. In this manner, the Anti-CD3 antibody is coated on an inner wall of the culture container to promote the activation and proliferation of the memory lymphocytes.

According to embodiments of the present disclosure, the peripheral blood mononuclear cells are obtained by the following manners: mixing peripheral blood with heparin and centrifuging at 1,200 rpm/min to 2,000 rpm/min for 5 to 10 minutes to obtain an upper layer of autologous plasma and a lower layer of blood cells; and diluting the blood cells with normal saline, loading the diluted blood cells on a surface of a lymphocyte separation solution, centrifuging the lymphocyte separation solution at 1,500 rpm/min to 2,000 rpm/min for 20 to 30 minutes, taking a mononuclear cell layer, mixing the mononuclear cell layer with the normal saline, centrifuging at 1,500 to 2,000 rpm/min for 5 to 10 minutes, and washing the centrifuged mononuclear cell layer for 3 times, so as to obtain the peripheral blood mononuclear cells, in which a volume ratio of the blood cells, the normal saline and lymphocyte separation liquid is (1 to 3):(1 to 3):1. Therefore, the peripheral blood mononuclear cell obtained has a high yield, high purity and strong activity.

A person skilled in the art can understand that features and advantages described

above in terms of the culture medium are also applicable to the method for obtaining the memory lymphocyte population, and will not be repeated here.

### Memory Lymphocyte Population

In another aspect of the present disclosure, the present disclosure provides a memory lymphocyte population. According to embodiments of the present disclosure, the memory lymphocyte population is obtained by the aforementioned method for obtaining the memory lymphocyte population. The memory lymphocyte population according to embodiments of the present disclosure can be activated, proliferate and differentiate rapidly under specific stimulation of liver cancer antigens, and can secrete IFN-γ, thereby assisting in strengthening the body immune response. In addition, since the memory lymphocyte population can be retained in the body, it has a better tumor killing effect. Also, the memory lymphocyte population can effectively reduce the risk of tumor metastasis and recurrence, and is especially suitable for liver cancer with microvascular invasion.

A person skilled in the art can understand that features and advantages described above in terms of the method for obtaining the memory lymphocyte population are also applicable to the memory lymphocyte population, and will not be repeated here.

### Use of a Memory Lymphocyte Population in a Preparation of a Medicine

In another aspect of the present disclosure, the present disclosure provides a use of the aforementioned memory lymphocyte population in a preparation of a medicine. According to embodiments of the present disclosure, the medicine is used to treat liver cancer. The memory lymphocyte population according to embodiments of the present disclosure can be activated, proliferate and differentiate rapidly under specific stimulation of liver cancer antigens, and can secrete IFN-γ, thereby assisting in strengthening the body immune response. In addition, since the memory lymphocyte population can be retained in the body, it has a better tumor killing effect. In addition, the memory lymphocyte population can effectively reduce the risk of tumor metastasis and recurrence, and is especially suitable for liver cancer with microvascular invasion. The memory lymphocyte population can assist tumor resection surgery, radiotherapy or chemotherapy treatment, or the memory lymphocyte population can be used in combination with an immune checkpoint inhibitor to assist the tumor resection surgery, radiotherapy or chemotherapy, so as to achieve a therapeutic purpose.

According to embodiments of the present disclosure, the medicine reduces a volume of a tumor tissue. As a result, the medicine has a better tumor killing effect. In addition, the medicine can effectively reduce the risk of tumor metastasis and recurrence, and is especially suitable for liver cancer with microvascular invasion.

According to embodiments of the present disclosure, the medicine reduces a content of alpha-fetoprotein in a body administrated with the medicine. As a result, the medicine has a better tumor killing effect. In addition, the medicine can effectively reduce the risk of tumor metastasis and recurrence, and is especially suitable for liver cancer with microvascular invasion.

According to embodiments of the present disclosure, the medicine has no hepatic and renal toxicity to the body administrated with the medicine.

It should be noted that the term “treatment” used in the present disclosure refers to obtaining a desired pharmacological and/or physiological effect. The effect can be preventive in terms of completely or partially preventing a disease or a symptom of the disease, and/or can be therapeutic in terms of partially or completely curing a disease and/or an adverse effect caused by the disease. The term “treatment” as used herein is directed to a disease of a mammal, especially human, and includes: (a) prevention of a disease (for example, prevention of liver cancer) or an occurrence of the disease in an individual who is susceptible to the disease but not yet diagnosed; (b) inhibiting the disease, for example, retarding the development of the disease; or (c) alleviating the disease, for example, reducing symptoms associated with the disease. The “treatment” as used herein covers the administration of a drug or a compound to an individual to treat, cure, alleviate, ameliorate, mitigate, or inhibit the disease of the individual, and includes, but not limited to, administering a medicine containing the memory lymphocyte population according to the present disclosure to an individual in need.

According to embodiments of the present disclosure, the memory lymphocyte population according to the present disclosure may be used in combination with a conventional treatment method and/or therapy, or may be used independently from the conventional treatment method and/or therapy. When the memory lymphocyte population according to the present disclosure is administered in a combination therapy with other medicines, the memory lymphocyte population may be administrated to the individual sequentially or simultaneously. Alternatively, the medicine according to the present disclosure may include a combination of the memory lymphocyte population according to the present disclosure, a pharmaceutically acceptable carrier or a pharmaceutically acceptable excipient, and other therapeutic or preventive medicines known in the art.

The term “administration” as used herein refers to an introduction of a predetermined amount of a substance into a patient in a suitable manner. The memory lymphocytes according to the present disclosure may be administered in any common manner as long as they can reach the desired tissue. Various routes of administration are expectable, including peritoneal, intravenous, intramuscular, subcutaneous, cortical, topical, nasal, lung, and rectal manners. However, the present disclosure is not limited to the exemplified routes of administration.

A person skilled in the art can understand that features and advantages described above for the memory lymphocyte population are also applicable to the use of the memory lymphocyte population in a preparation of a medicine, and will not be repeated here.

The solutions of the present disclosure will be explained below in combination with examples. A person skilled in the art will understand that the following examples are only used to illustrate the present disclosure and should not be regarded as limiting the scope of the present disclosure. Where specific techniques or conditions are not indicated in the examples, procedures shall be carried out in accordance with techniques or conditions described in the literature in the art or in accordance with a product specification. Reagents or instruments used without specifying manufacturers are those conventional and commercially available.

### Example 1

In this example, memory lymphocytes are obtained according to the following method:

### 1. Culture Medium

GT-T551 culture medium, 5×10⁵ U/L of interleukin-2, 5 ng/mL of interleukin-7, 5 ng/mL of interleukin-15, 3 μg/mL of Anti-CD3 antibody, 5 vol % of autologous plasma, a pH value of 7.2 to 7.4.

A culture flask was coated with the Anti-CD3 antibody in advance. A volume of a coating solution was 5 ml/75 cm² of the culture flask, and the coating solution was placed overnight at 4° C.

TABLE 1
Table of raw materials

| Reagent name | Brand |
|---|---|
| GT-T551 | Takara Bio |
| Interleukin-2 | Jiangsu Kingsley |
| Interleukin-7 | R&D Systems |
| Interleukin-15 | R&D Systems |
| Anti-CD3 antibody | ACROBiosystems |

### 2. Culture Method

(1) Heparin was used as an anticoagulant to extract fresh peripheral blood donated by a liver cancer patient. The fresh peripheral blood was centrifuged at 1,600 rpm/min for 5 to 10 minutes to extract autologous plasma, a lower layer of which was blood cells. The autologous plasma was inactivated at a temperature of 56° C. for 30 minutes and used to prepare a culture medium.
(2) The blood cells were diluted with normal saline and added dropwise to a lymphocyte separation solution. A ratio of the blood cells, normal saline, and lymphocyte separation solution was 1:1:1;

The blood cells diluted with the normal saline were centrifuged at 1,500 rpm/min to 2,000 rpm/min for 20 to 30 min to obtain a mononuclear cell (PBMC) layer. The mononuclear cell layer was centrifuged at 1,500 rpm/min to 2,000 rpm/min for 5 to 10 min in saline, and washed 3 times to obtain peripheral blood mononuclear cells.
(3) The peripheral blood mononuclear cells were suspended in the culture medium at a density of 5×10⁵ cells/mL to 20×10⁵ cells/mL, and cultured at a temperature of 37° C. and with 5% carbon dioxide for 14 days. A cell density was relatively high and a color of the culture medium changed to yellow. The suspended cells were resuspended in the culture medium for subculture.

During the culturing, a fresh medium was added every 2 to 4 days to maintain a cell density of 5×10⁵ cells/mL to 25×10⁵ cells/mL. The autologous plasma was added during vaccination and the first and second supplementations, and the autologous plasma was not required for the latter two times of rehydration.

### Example 2: Evaluation Test of Curative Effect of Memory Lymphocytes on Transplanted Liver Cancer in a Nude Mice

In this example, tumor antigens derived from SMMC-7721 cells were selected to observe differentiation, proliferation and effect ability of the memory lymphocytes under a stimulation of DC cells loaded with the tumor antigens and to provide reference for a relevant mechanism of clinical applications.

### 1. Materials and Methods

The DC cells used in an experimental examples were obtained from an adherent part of PBMCs of peripheral blood of a healthy human body under culture conditions that the adherent part contained GM-CSF, interleukin-4 and TNF-α, and were identified as CD45⁺CD11b⁺CD11c⁺ by flow cytometry.

The tumor antigens derived from SMMC-7721 cells used in the experimental examples were obtained by dissolving a SMMC-7721 cell line repeatedly frozen and thawed in liquid nitrogen in 0.1% HCLO (normal saline as a solvent).

### 2. Steps of the Experiments

(1) On the 6-th day of DC culturing, a solution dissolving the tumor antigens derived from SMMC-7721 was added to the culture medium. On the 7-th day, the DC cells loaded with the tumor antigens derived from SMMC-7721 were harvested and identified as CD45⁺CD11b⁺CD11c⁺HLA-DR⁺ by flow cytometry.
(2) PBMCs from the peripheral blood of the patient with liver cancer were taken and cultured in accordance with the protocol in Example 1. After 14 days, liver cancer-specific memory lymphocytes were obtained, and an expression of specific T cell marker CD39 and an expression of failure T cell marker PD-1 were detected by flow cytometry.
(3) The above-mentioned DC cells and CD3⁺CD45RA⁻45RO⁺CD62L⁺ memory lymphocytes were co-cultured at a ratio of 1:5 for 72 hours, and then a change in CD62L expression on a surface of the memory lymphocytes was detected by flow cytometry.
(4) The above-mentioned DC cells and CFSE-stained CD3⁺CD45RA⁻ CD45RO⁺CD62L⁺ memory lymphocytes were co-cultured at a ratio of 1:5 for 72 hours, and then flow cytometry was used to detect a decrease in a fluorescence intensity of CFSE in the memory lymphocytes, namely, proliferation of the memory lymphocytes.
(5) The above-mentioned DC cells and CD3⁺CD45RA⁻CD45RO⁺CD62L⁺ memory lymphocytes were co-cultured at a ratio of 1:5 for 72 hours, and an expression of an effector cytokine IFN-γ in the memory lymphocytes was detected by flow cytometry.

### 3. Results of the Experiments

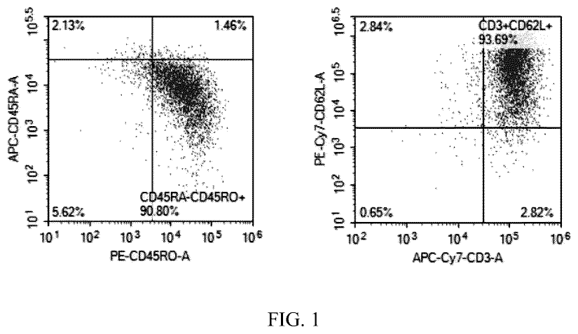

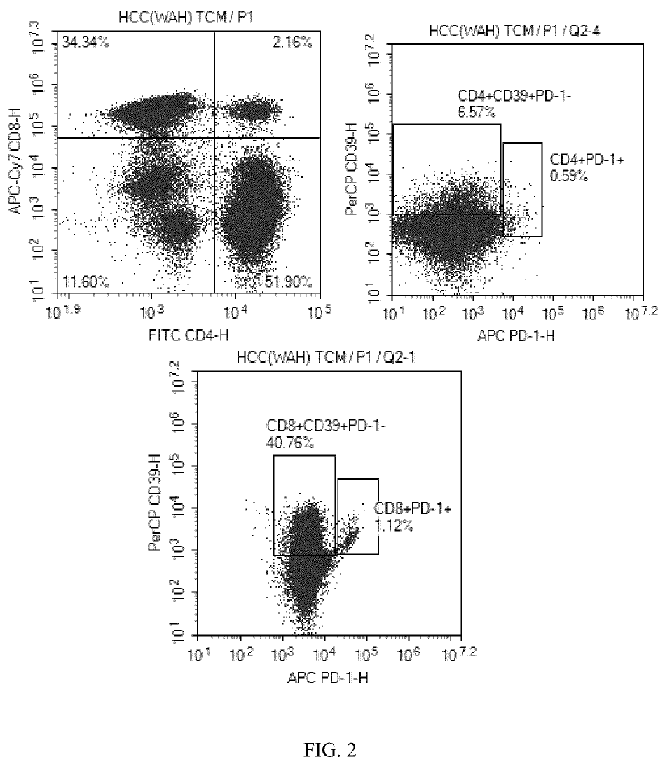

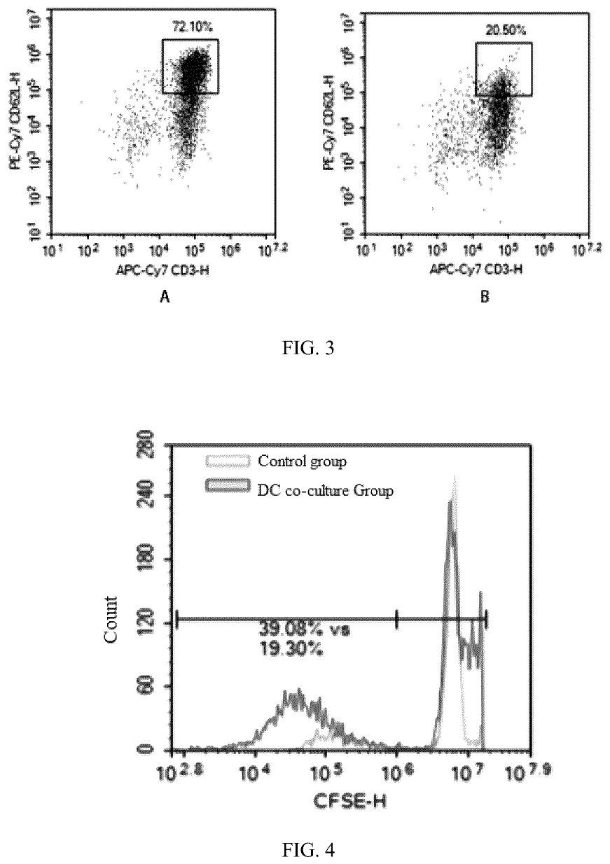

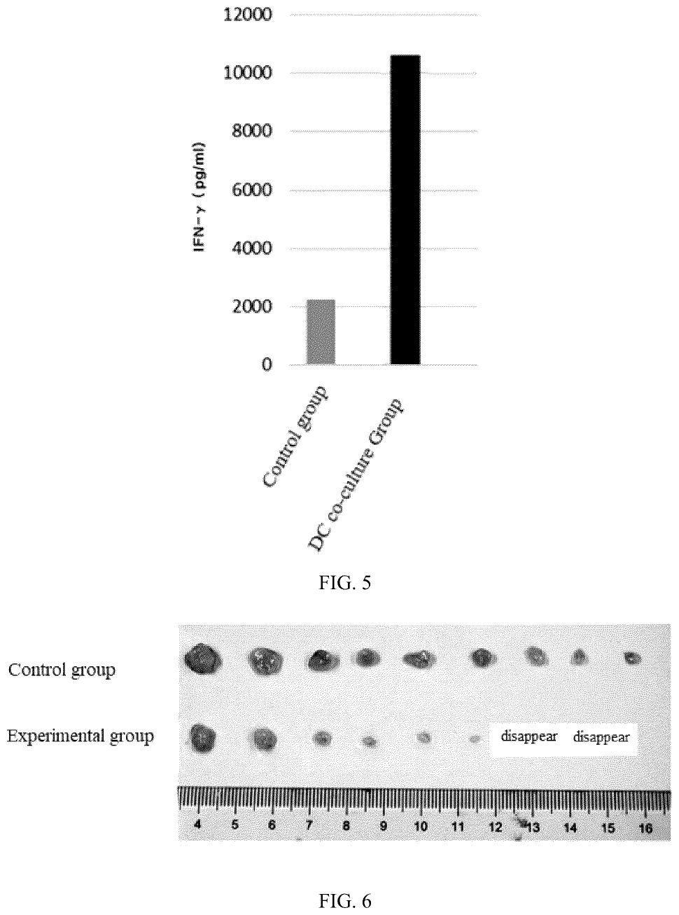

(1) A main cell population of the memory lymphocytes in this example is central memory T cells, and a surface marker molecule is CD3⁺CD45RA⁻CD45RO⁺CD62L⁺ (FIG. 1). A test found that proportions of CD39⁻PD-1⁻ cell subset in CD4⁺ and CD8⁺ central memory T cells were 6.57% and 40.76%, respectively, indicating that memory lymphocytes related to tumor-specific killing were mainly derived from CD8⁺ T cells (FIG. 2).
(2) In this example, after the memory lymphocytes were co-cultured with the DC cells loaded with the tumor antigens derived from SMMC-7721, a proportion of CD62L⁺ cells was significantly reduced, and a proportion of CD62L⁻ cells was significantly increased (FIG. 3), indicating that under the stimulation of the antigens, the central memory lymphocytes differentiated into effector memory lymphocytes.
(3) In this example, after the CFSE-stained memory lymphocytes were co-cultured with the DC cells loaded with the tumor antigens derived from SMMC-7721, the fluorescence intensity of CFSE in some memory lymphocytes was significantly reduced. Compared with a simple memory lymphocyte culture group, a proportion of proliferating cells in the same culture group with DC was roughly doubled (19.3% vs. 39.08%) (FIG. 4).
(4) In this example, after the DC cells loaded with the tumor antigens derived from SMMC-7721 were co-cultured with the memory lymphocytes, the expression of the effector cytokine IFN-γ in the memory lymphocytes was significantly increased (FIG. 5).

### Example 3: Evaluation Test of Curative Effect of Memory Lymphocytes on Transplanted Liver Cancer in Nude Mice

In this example, SMMC-7721 cells were selected to establish a nude mouse xenograft tumor model, to observe a growth inhibitory effect of the memory lymphocytes on the nude mouse xenograft tumor, and to provide reference for clinical applications.

### 1. Materials and Methods

A test product (memory lymphocyte fluid) used in this example was a colorless and transparent liquid with a cell content/specification of 5×10⁷/mL, and a ratio of CD3⁺ CD45RA⁻CD45RO⁺CD62L⁺ cells was not less than 70%.

Experimental animals used in this example were purchased from Beijing Vital River Laboratory Animal Technology Co., Ltd., SPF-grade BALB/c nude mice, each weighing 16 to 18 g, 6 to 7 weeks old. A total of 20 female mice was purchased and divided into two groups for experimental use.

The grouping was conducted as follows. The animals were weighed on the last day of a quarantine period, and randomly divided into two groups according to their weights. Each group included 10 animals, as illustrated in Table 2. If necessary, after the tumor model was established, animals that had not been infused were grouped again according to tumor volumes.

TABLE 2
Test group

| No. | Group | Number | Animal No. and sex |
|---|---|---|---|
| 1 | Tumor model group | 10 | 01-10/♀ |
| 2 | Intravenous infusion group | 10 | 11-20/♀ |
| | after tumor formation | | |

### 2. Steps of the Experiments

### (1) Establishment of SMMC-7721 Nude Mouse Xenograft Tumor Model

SMMC-7721 cells in an exponential growth stage were collected under aseptic conditions. The cell density was adjusted to 1×10⁷ cells/mL with sterile normal saline to prepare a single cell suspension (the single cell suspension is produced by Beijing Jingyuan Yaneng Biotech Co., Ltd.). The cell suspension was transported in a cold chain during transportation and stored in ice water before inoculation. At the time of inoculation, 0.1 ml of the single cell suspension (5×10⁶ cells/mouse) was taken and subcutaneously injected into an armpit of a forelimb of a nude mouse. The establishment of the tumor model was continuously observed.

### (2) Administration Plan

Except for the tumor model group, all other groups were infused with the memory lymphocytes on the 7th, 23rd, 26th, and 30th day after tumor inoculation. Administration route, period and frequency are shown in Table 3.

TABLE 3
Administration plan

| | Infusion dose | Administration | First | Times of |
|---|---|---|---|---|
| Group | (cells/mouse) | route | administration | administration |
| 1. Tumor model group | — | — | — | — |
| 2. Intravenous infusion | 5 × 10⁶ | Tail vein | Tumor grows to | 4 times |
| group after tumor | | | 3 mm | |
| formation | | | | |

### (3) Detection Frequency and Method of Various Indicators

Detections of various indicators were divided into: observation by the cage, weight measurement, and tumor measurement.

### (a) Observation by the Cage

Observation frequency and time: 1 time per day.

Cases of observations: all animals.

Observation method: observing tumor formation at a transplantation site of a nude mouse, and death cases.

### (b) Weight Measurement

Number and time of measurement: twice a week.

Cases of measurement: all animals.

Measurement method: using sartorius electronic balance for weighing.

### (c) Tumor Measurement

Number and time of measurement: the measurement was started from the day the tumor was found. Before starting the administration, the measurement was conducted three times a week.

Cases of measurement: all animals.

Measurement method: using a vernier caliper to measure long and short diameters of tumors on the experimental animals.

### (d) Detection of Infiltrating T Cells in a Tumor

Number and time of measurement: detection was conducted after sacrificing tumor-bearing mice.

Cases of measurement: some animals.

Measurement method: immunohistochemical staining on paraffin sections of tumor tissues (Anti-Human CD8)

### (e) Detection of Peripheral Blood Retention After Reinfusion of the Memory Lymphocytes

Number and time of measurement: detection was conducted immediately after killing tumor-bearing mice.

Cases of measurement: some animals.

Measurement method: using flow cytometer to analyze a proportion of memory T cells and a proportion of TCM cells in the peripheral blood of mice.

### (4) Result Statistics and Analysis

### (a) Calculation of Tumor Volume

The tumor volume (TV) was calculated according the following equation:

TV−a×b²/2 (where a is a long diameter of the tumor and b is a short diameter of the tumor).

### (b) Statistical Method

For measurement data such as body weight, tumor volume, tumor weight, etc., statistics was performed based on the following methods:
(i) First, the One-Sample Kolmogorov-Smirnov Test was used to test whether the data conform to a normal distribution. The Levene's median method was used to test the homogeneity of variance, and a one-way analysis of variance (One-Way ANOVA) method was adopted. If tests for normality and homogeneity of variance failed, a non-parametric Kruskal-wallis test was required.
(ii) If a test result of a variance analysis was significant (P≤0.05), Dunnett's test was further used for multiple comparison tests. If the result of the test analysis of variance was not significant (P>0.05), the statistics was ended.
(iii) If a Kruskal-wallis test result was significant (P≤0.05), Mann-Whitney test was further used for multiple comparison tests. If the Kruskal-wallis test result was not significant (P>0.05), the statistics was ended.

### 3. Experimental Results

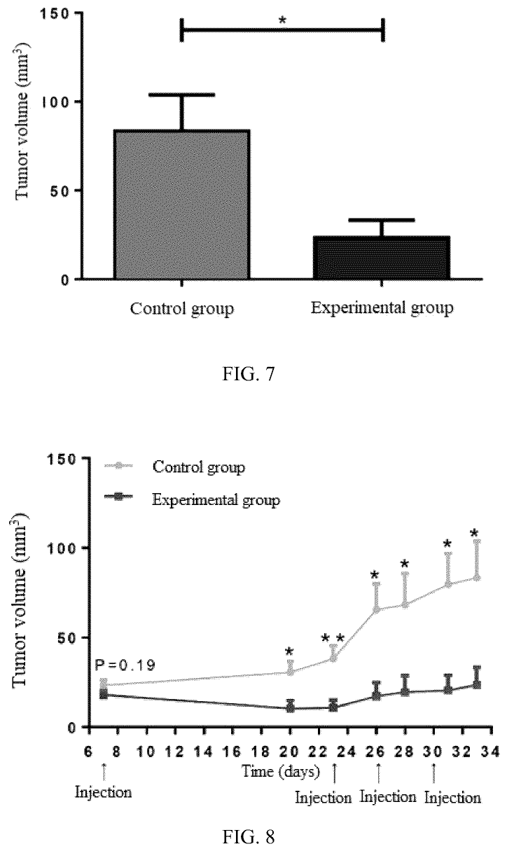

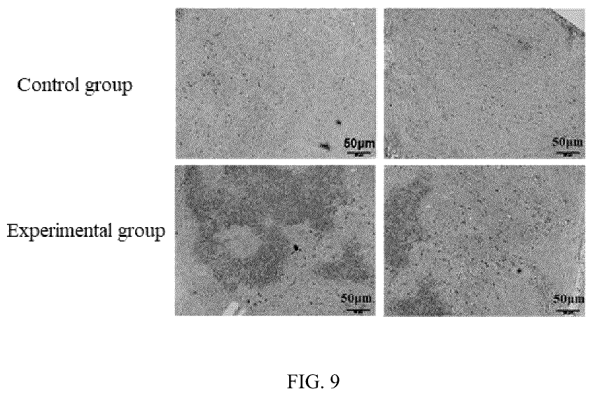

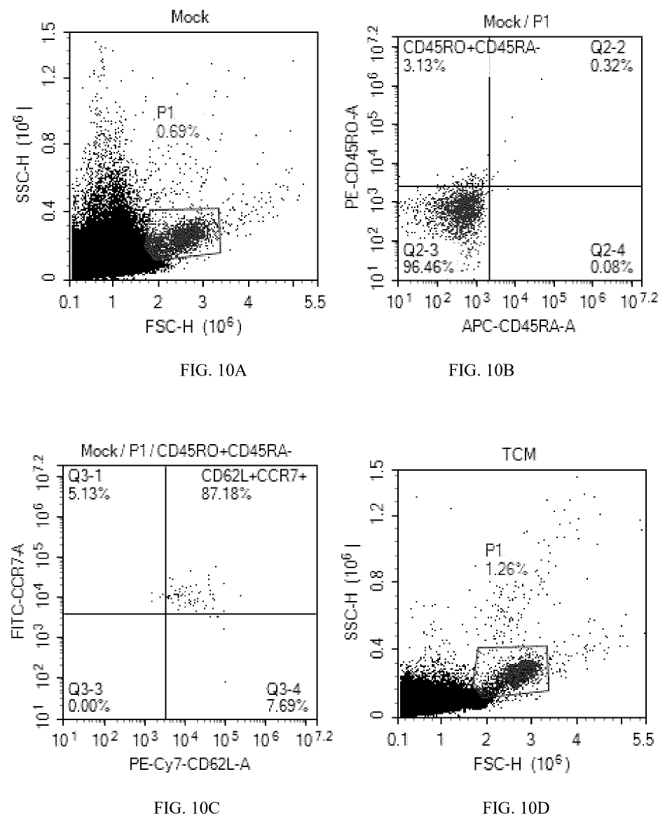

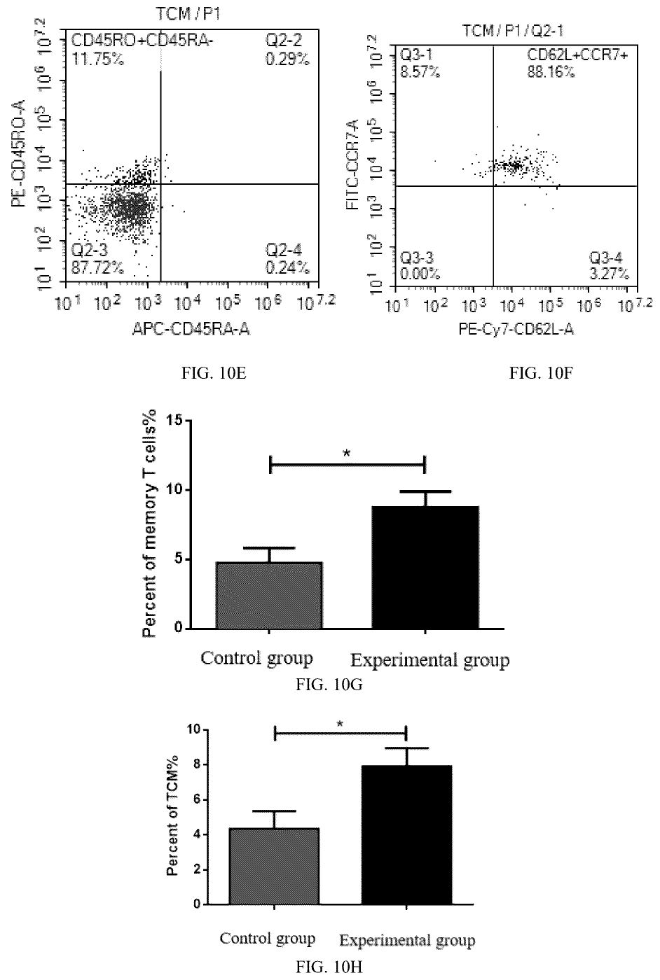

From the day of tumor formation, the tumor volume was measured twice a week in the first 1-5 weeks. It was found that tumor volumes of the control group (with infusion of normal saline) continued to increase, while tumor volumes of the experimental group (with continuous infusion of the memory lymphocytes) continued to decrease. Tumors in some mice even disappeared in the 4th week after treatment (FIG. 6). In the 5th week, the tumor volumes of the experimental group and the control group were statistically analyzed. It was found that an average tumor volume of mice in the control group was close to 100 mm³, and an average tumor volume of mice in the experimental group was smaller than 25 mm³ (FIG. 7). From follow-up data, the volumes of tumors borne by the mice decreased significantly from the 3rd week after the infusion of the memory lymphocytes, and were maintained within a range of smaller than 25 mm³ (FIG. 8). The tumor-bearing nude mice were sacrificed to take the tumors out for immunohistochemical staining. The darker the brown color was, the more CD8⁺ T cells were infiltrated. The results show that a proportion of human-derived CD8⁺ T cell infiltration in the tumor tissues of the experimental group was significantly higher than that in the control group (FIG. 9). After the tumor-bearing mice were sacrificed, proportions of human memory T cells in the peripheral blood of the mice were detected at the same time. The results indicate that a ratio of the memory T cells (CD3 CD45RO⁺ cells) and a ratio of TCM in the experimental group were significantly higher than those in the control group. In addition, TCM accounts for more than 85% of T cells, confirming that the memory lymphocytes infused back can survive in the mice and exert anti-tumor effects (FIG. 10).

### Example 4: Treatment of Patients With Liver Cancer by Using the Memory Lymphocytes to Assist Tumor Resection Operations

This example is a multi-center randomized controlled study of the memory

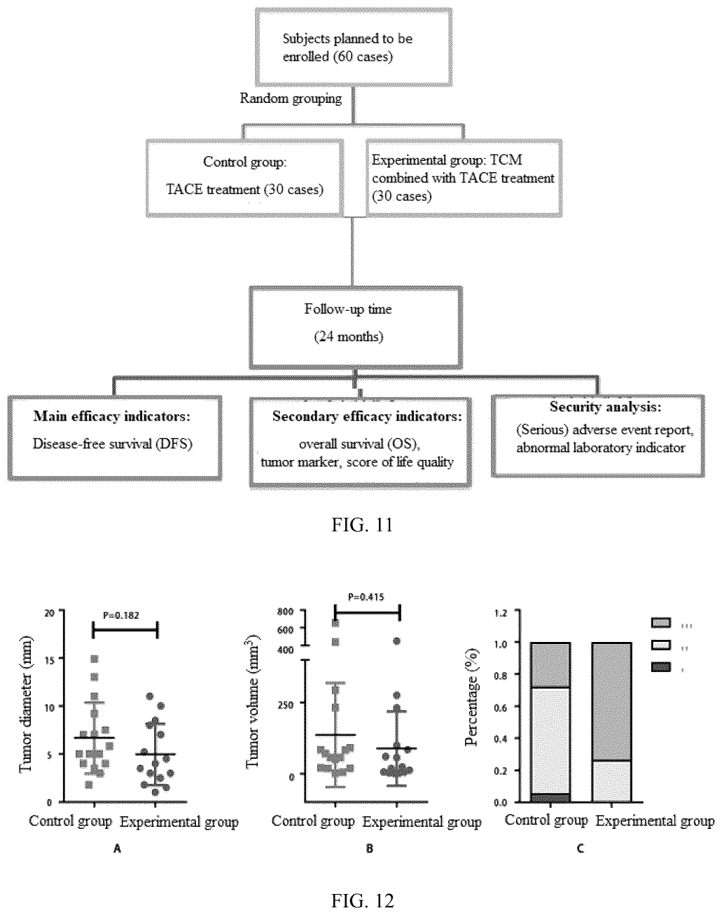

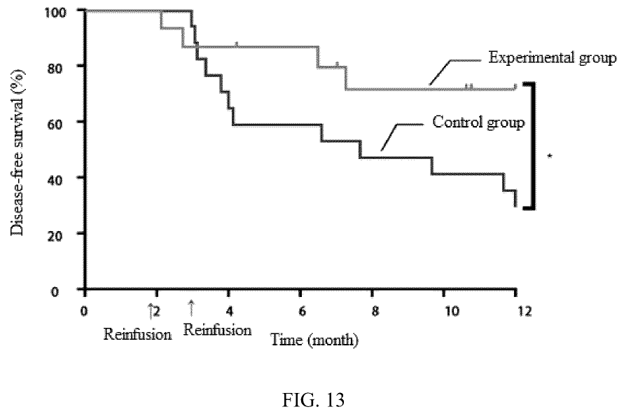

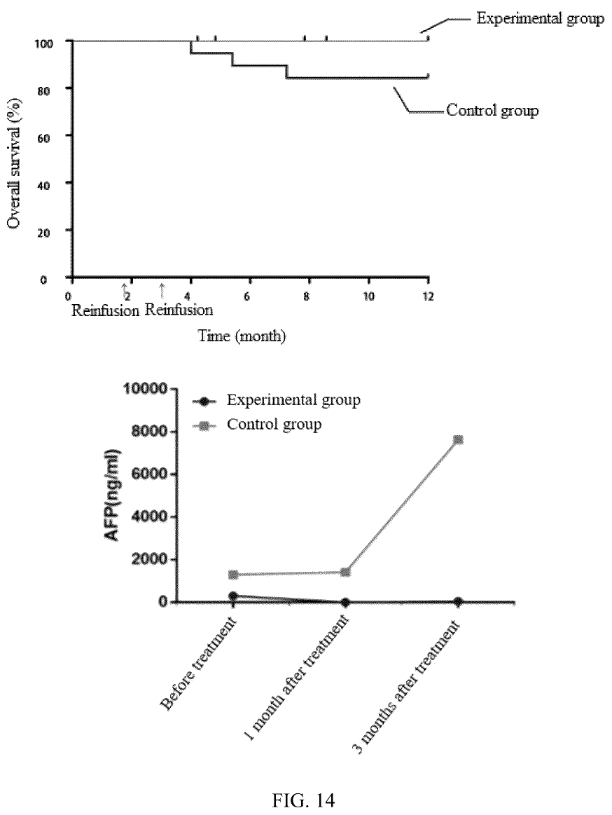

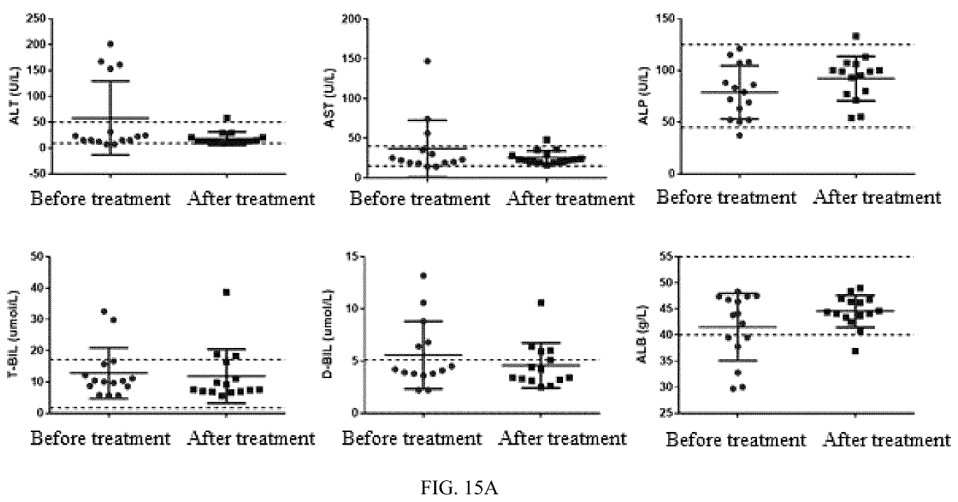

lymphocytes combined with transhepatic arterial infusion chemoembolization (TACE) in patients with microvascular invasion (MVI) after radical resection operations of liver cancer, and this study has passed the review of Ethics Committee of Cancer Hospital, Chinese Academy of Medical Sciences
(1) Research objective: to evaluate clinical efficacy and safety of TCM cell therapy in patients with the microvascular invasion (MVI) after radical resection operations of liver cancer.
(2) Enrolling criteria:
   Undergoing radical hepatectomy, preoperative imaging did not indicate vascular invasion
   Postoperative pathology confirmed complete resection of the tumor, and the tissue type was hepatocellular carcinoma with MVI under microscope
   Eligibility scan confirmed CR after independent radiology review
   Child-Pugh Grade A (<7 scores)
   ECOG stamina score was 0 or 1
(3) Exclusion criteria:
   Patients with recurrent hepatocellular carcinoma
   Portal vein tumor thrombus
   in process of or long-term use of an immunosuppressive drug
   Severe coagulation abnormality
   Severe leukopenia or bone marrow transplant
   Patients with severe liver, kidney or heart failure
   Patients with uncontrolled severe infection or high fever
   Patients with a severe autoimmune disease
   Patients with intractable or persistent epilepsy
(4) Refer to FIG. 11 for the technical route and research endpoint.

Referring to Table 3, 22 subjects have been enrolled in the control group, and 5 patients have been removed from the group, in which 2 patients miss the follow-up, 1 patient was re-enrolled in the group, and 2 patients have relapsed before the first interventional treatment after radical operation. In the experimental group, 18 patients have been enrolled, and 3 patients have been removed from the group, in which 1 patient voluntarily asked to leave before cell therapy, 1 patient was diagnosed with primary colorectal cancer after enrollment, and 1 patient has relapsed before the first cell reinfusion after a radical operation of liver cancer.

TABLE 3
Enrollment of clinical trials

| | | Memory lymphocyte |
|---|---|---|
| | TACE treatment group | combined treatment group |
| Total enrolled cases, N | 22 cases | 18 cases |
| Cases removed from | 5 cases | 3 cases |
| the group, N | | |
| Enrolled subjects, N | 17 cases | 15 cases |
| Underlying diseases | 16 cases of HBV positive, and | 14 cases of HBV positive, and |
| | 1 case of HBV negative | 1 case of HBV negative |
| Lymphatic or distant | None | None |
| metastasis, N | | |
| Number of lesions | One: 14 cases (82.3%) | One: 13 cases (86.7%) |
| | Two: 2 cases (11.8%) | Two: 2 cases (13.3%) |
| | Three: 1 case (0.06%) | |

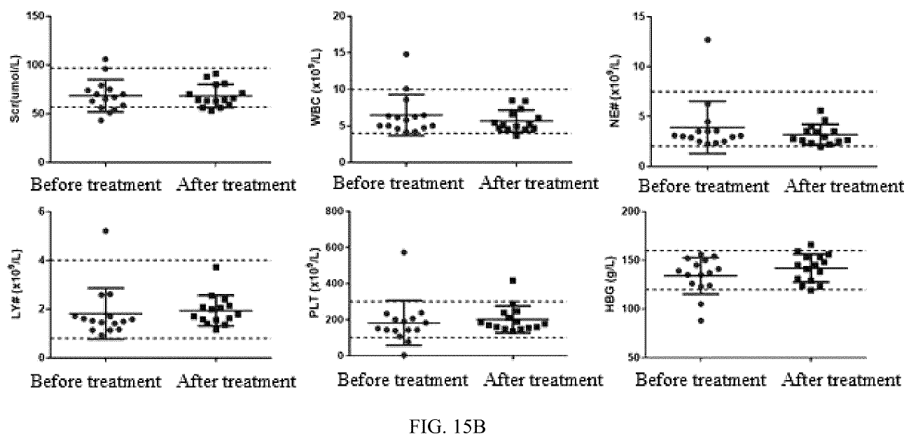

The results show that, when follow up the patients to 12 months, compared with TACE therapy alone, memory lymphocytes combined with TACE therapy can prolong a median disease-free survival (mDFS, the group with the memory lymphocytes combined therapy>12 months, the group with TACE therapy alone=7.67 months, P=0.046), reduce a tumor recurrence rate (26.6% vs 70.6%), and significantly reduce a mortality (0% vs 17.6%) and a level of in vivo tumor marker AFP of the subjects. Preliminary statistical results find that the safety of memory lymphocyte-related therapy is good, and there is no obvious liver and kidney damage (see FIG. 15).

Reference throughout this specification to “an embodiment,” “some embodiments,” “an example,” “a specific example,” or “some examples,” means that a particular feature, structure, material, or characteristic described in connection with the embodiment or example is included in at least one embodiment or example of the present disclosure. The above phrases in various places throughout the specification are not necessarily referring to the same embodiment or example of the present disclosure. Furthermore, the particular features, structures, materials, or characteristics can be combined in any suitable manner in one or more embodiments or examples. In addition, different embodiments or examples and features of different embodiments or examples described in the specification can be combined by those skilled in the art without mutual contradiction.

Although embodiments of present disclosure are illustrated and described above, it should be understood that above embodiments are merely illustrative, and cannot be construed to limit the present disclosure. Those skilled in the art can make changes, alternatives, and modifications to the embodiments without departing from spirit, principles and scope of the present disclosure.

What is claimed is:

1. A method for treating liver cancer, the method comprising:
administrating a therapeutic effective amount of a memory lymphocyte population to a subject in need thereof,
wherein the memory lymphocyte population comprises at least one of leukocyte differentiation antigens CD3, CD4, CD8, CD16, CD56, CD62L, or CD45RO.

2. The method according to claim 1, wherein:
a main cell population in the memory lymphocyte population is central memory T cells;
a content of the main cell population is not less than 70%; and
a surface marker molecule of the main cell population is CD3⁺ CD45RA⁻ CD45RO⁺CD62L⁺.

3. The method according to claim 2, wherein a proportion of CD39⁺PD-1⁻ cell subset in the central memory T cells with a surface marker molecule of CD4⁺ is 5% to 8%.

4. The method according to claim 2, wherein a proportion of CD39⁺PD-1⁻ cell subset in the central memory T cells with a surface marker molecule of CD8⁺ is 35% to 45%.

5. The method according to claim 1, wherein when the memory lymphocyte population is in contact with DC cells loaded with tumor antigens, a proportion of cells with a surface marker molecule of CD62L⁺ in the memory lymphocyte population decreases, a proportion of cells with a surface marker molecule of CD62L⁻in the memory lymphocyte population increases, and an expression of IFN-γ increases.

6. The method according to claim 1, further comprising, for the subject who has undergone radical hepatectomy:
subjecting the subject to transhepatic arterial infusion chemoembolization, TACE, therapy.

7. The method according to claim 1, further comprising, prior to said administrating a therapeutic effective amount of a memory lymphocyte population to a subject in need thereof:
resuspending and culturing naive immune cells in a culture medium, to obtain the memory lymphocyte population,
wherein the culture medium comprises:
a basal culture medium;
interleukin-2;
interleukin-7;
interleukin-15;
an Anti-CD3 antibody; and
autologous plasma.

8. The method according to claim 7, wherein said culturing is performed at a temperature of 37° C. and 5 vol % of CO₂ for 10 to 20 days.

9. The method according to claim 7, wherein the culture medium is supplemented every 2 to 4 days during said culturing until a cell density ranges from 5×10⁵ cells/mL to 25×10⁵ cells/mL, wherein the culture medium does not contain the autologous plasma in a third supplementation and each of subsequent supplementations.

10. The method according to claim 7, wherein the naive immune cells are selected from peripheral blood mononuclear cells.

11. The method according to claim 10, wherein the peripheral blood mononuclear cells are resuspended in the culture medium at a density of 5×10⁵ cells/mL to 20×10⁵ cells/mL.

12. The method according to claim 10, wherein the peripheral blood mononuclear cells are obtained by:
mixing peripheral blood with heparin and centrifuging at 1,200 rpm/min to 2,000 rpm/min for 5 to 10 minutes to obtain an upper layer of autologous plasma and a lower layer of blood cells; and
diluting the blood cells with normal saline, loading the diluted blood cells on a surface of a lymphocyte separation solution, centrifuging the lymphocyte separation solution at 1,500 rpm/min to 2,000 rpm/min for 20 to 30 minutes, taking a mononuclear cell layer, mixing the mononuclear cell layer with the normal saline, centrifuging at 1,500 rpm/min to 2,000 rpm/min for 5 to 10 minutes, and washing the centrifuged mononuclear cell layer for 3 times, so as to obtain the peripheral blood mononuclear cells,
wherein a volume ratio of the blood cells, the normal saline and lymphocyte separation liquid is (1 to 3):(1 to 3):1.

13. The method according to claim 7, further comprising, prior to said resuspending and culturing naive immune cells in a culture medium:
coating a culture container with a coating solution containing the Anti-CD3 antibody at 2° C. to 8° C. for 10 to 16 hours, wherein a volume of the coating solution is 2 to 8 ml/75cm² of the culture container.

14. The method according to claim 7, wherein in the culture medium:
a concentration of the interleukin-2 is 5×10⁴ U/L to 1×10⁶ U/L;
a concentration of the interleukin-7 is 1 ng/mL to 60 ng/mL;
a concentration of the interleukin-15 is 1 ng/mL to 60 ng/ml;
a concentration of the Anti-CD3 antibody is 0.5 μg/mL to 10 μg/mL; and
a concentration of the autologous plasma is 1 vol % to 10 vol %.

15. The method according to claim 7, wherein in the culture medium:
a concentration of the interleukin-2 is 5×10⁵ U/L;
a concentration of the interleukin-7 is 5 ng/ml;
a concentration of the interleukin-15 is 5 ng/ml;
a concentration of the Anti-CD3 antibody is 3 μg/mL; and
a concentration of the autologous plasma is 5 vol %.

16. The method according to claim 7, wherein a pH value of the culture medium ranges from 7.2 to 7.4.

17. A method for treating liver cancer in assistance with tumor resection operation, the method comprising:
administrating a therapeutic effective amount of a memory lymphocyte population to a who has undergone radical hepatectomy; and
subjecting the subject to TACE therapy,
wherein the memory lymphocyte population comprises at least one of leukocyte differentiation antigens CD3, CD4, CD8, CD16, CD56, CD62L, or CD45RO.

18. The method according to claim 17, wherein a main cell population in the memory lymphocyte population is central memory T cells;
a content of the main cell population is not less than 70%; and
a surface marker molecule of the main cell population is CD3⁺CD45RA⁻ CD45RO⁺CD62L⁺.

19. The method according to claim 18, wherein a proportion of CD39⁺PD-1⁻ cell subset in the central memory T cells with a surface marker molecule of CD4⁺ is 5% to 8%.

20. The method according to claim 18, wherein a proportion of CD39⁺PD-1⁻ cell subset in the central memory T cells with a surface marker molecule of CD8⁺ is 35% to 45%.
